(19) United States
(12) Patent Application Publication
(10) Pub. No.: US 2024/0084254 A1
(43) Pub. Date: Mar. 14, 2024

(54) CELL PRESERVATION SOLUTION, PREPARATION METHOD FOR SAME, AND APPLICATIONS THEREOF

(71) Applicant: JIANGSU RENOCELL BIOTECH CO., LTD., Nanjing (CN)

(72) Inventor: Guangji Wang, Nanjing (CN)

(73) Assignee: JIANGSU RENOCELL BIOTECH CO., LTD., Nanjing (CN)

(21) Appl. No.: 18/513,797

(22) Filed: Nov. 20, 2023

Related U.S. Application Data
(63) Continuation of application No. PCT/CN22/75606, filed on Feb. 9, 2022.

(30) Foreign Application Priority Data
Mar. 20, 2021 (CN) CN202110298828.X

(51) Int. Cl. C12N 5/0783; A01N 1/02

(52) U.S. Cl. CPC C12N 5/0636; A01N 1/0226; A01N 1/0284

(57) ABSTRACT

Disclosed are a cell preservation solution, a preparation method for same, and applications thereof. The present disclosure specifically relates to a mesenchymal stem cell, NK cell, and CART cell preservation solution, a preparation method for same, and applications thereof. The contents of the components of the cell preservation solution are as follows: every 100 ml of the cell preservation solution contains 5-15 ml of human serum albumin, 80-90 ml of a compound electrolyte injection, 1-5 ml of a sulfate antioxidant-free compound amino acid injection, 0.01-1 g of L-glutamine, and 0.1-1 g of anhydrous glucose. The preservation solution of the present disclosure maintains a cell survival rate of no less than 80% for 144 h or more and an agglomeration rate of less than 12%, extends to the greatest extent the preservation time of a cell preparation, and implements the possibility for application at the national and even the global level. All of the components applied in the present disclosure are clinical-grade medicaments and are safe and reliable for clinical applications.

### FIELD

The present disclosure belongs to the technical field of cell preservation, and relates to a cell preservation solution and a preparation method and application thereof. Specifically, it relates to a cell preservation solution for mesenchymal stem cells, NK cells, CART cells, DC cells, CIK cells, PBMCs, and hematopoietic stem cells, and a preparation method and application thereof.

### BACKGROUND

In recent years, the continuous development of stem cell therapy, immune cell therapy and gene editing in basic theories, technical means and clinical medicine has enabled cell therapy to provide new treatment ideas and methods for some serious and refractory diseases. Cell therapy is a method of injecting living cells into patients to treat certain diseases. In recent years, the construction and development of cell banks and the resulting expansion of cell production and storage capabilities have enhanced the capacity of the global cell therapy market, and cell therapy products have thus received widespread attention.

Live cells are the primary active ingredients in cell therapy products. Maintaining high cell viability, total cell concentration, viable cell concentration, low cell clumping rate, and ensuring the clinical safety of the product are currently the main difficulties in clinical translation of cells. Therefore, there is an urgent need for a cell preservation solution to address the above problems.

The effective amount of cell therapy products is evaluated using three gold standards: viability rate, total cell concentration, and viable cell concentration. The relationship between these three can be expressed as viability rate=viable cell concentration/total cell concentration×100%. If both the viable cell concentration and total cell concentration fall below the quality control standard, the viability data may still meet the standards. Therefore, relying solely on a high viability rate indicator is insufficient to establish the effectiveness of cell therapy product dosage. However, current patents related to cell therapy products only display viability data without reflecting total cell concentration and viable cell concentration data. Thus, it cannot effectively prove that the product amount is effective.

The cell clumping rate indicates the degree of cell aggregation and is an important indicator for evaluating the safety of cell therapy products. In the existing technology, physiological saline supplemented with human serum albumin is often used as the cell preservation solution. During the transportation process, the cells can easily clump, causing great safety risks in intravenous infusion. When the cell clumping rate is high, injection into animals often causes fatal reactions such as pulmonary embolism and vascular embolism. Therefore, strict control of the clumping rate is necessary. In the existing technology, heparin is often added to prevent aggregation, but it is not suitable for patients with coagulation insufficiency.

Sulfites are often added as antioxidants in easily oxidizable components, but such antioxidants can elicit adverse responses in patients with sensitive constitutions, which can manifest as dermatitis, urticaria, flushing, hypotension, abdominal pain, diarrhea, etc. In severe cases, anaphylactic shock and asthma, which can be life-threatening, may develop. Patents pertaining to the prior art frequently involve the addition of ingredients containing such antioxidants, thereby engendering safety risks for clinical use.

Low-temperature transportation can not only slow down the metabolic rate of cells, but also avoid the safety risks associated with thawing, making it a superior transportation method to both normal temperature transportation and frozen transportation. However, there is currently no low-temperature preservation method and related reagents that can maintain high cell viability and viable cell concentration for a long time without any safety risks, thereby meeting national or even global requirements for cell transportation.

### SUMMARY

In order to overcome the above-mentioned shortcomings of the prior art, a primary object of the present disclosure is to provide a clinically safe and reliable cell preservation solution that maintains mesenchymal stem cells, NK cells, CART cells and other cells under low temperature conditions for up to 5 days or more with higher cell viability and viable cell concentration and low clumping rate.

Another object of the present disclosure is to provide a method for producing the above-mentioned cell preservation solution.

Another object of the present disclosure is to provide use of the cell preservation solution.

In order to achieve the above objects, the technical solutions adopted by the present disclosure are:

A composition for producing a cell preservation solution, consisting of a compound amino acid injection, L-glutamine and anhydrous glucose at a ratio of (0.1-5) ml:(0.01-1) g:(0.1-1) g.

As a preferred embodiment of the present disclosure, the compound amino acid injection is sulfite antioxidant-free compound amino acid injection 14AA, 14AA-SF.

Use of the composition of the present disclosure in the manufacture of a cell preservation solution.

A cell preservation solution comprising the composition of the present disclosure.

As a preferred embodiment of the present disclosure, the content of each component of the cell preservation solution is as follows: every 100 ml of the cell preservation solution comprises 5-15 ml of human serum albumin preparation, 80-90 ml of compound electrolyte injection, 0.1-5 ml of sulfite antioxidant-free compound amino acid injection, 0.01-1 g of L-glutamine, and 0.1-1 g of anhydrous glucose.

As a further preferable embodiment of the present disclosure, the sulfite antioxidant-free compound amino acid injection is sulfite antioxidant-free compound amino acid injection 14AA, 14AA-SF.

As a further preferable embodiment of the present disclosure, the osmotic pressure of the preservation solution is 275-345 mOsmol/kg.

As a further preferable embodiment of the present disclosure, the pH of the preservation solution is 6.0-8.0.

As a further preferable embodiment of the present disclosure, the original concentration of the human serum albumin preparation is 10 g/50 ml, which is an important substance for maintaining osmotic pressure.

As a preferred embodiment of the present disclosure, the specification of the compound electrolyte injection is 500 ml/bag, which is an important auxiliary ingredient for maintaining osmotic pressure and various biological functions.

As a preferred embodiment of the present disclosure, the sulfite antioxidant-free compound amino acid injection is sulfite antioxidant-free compound amino acid injection 14AA (14AA-SF). The compound amino acid can provide essential amino acids for cells and participate in the synthesis and metabolism of various substances.

The L-glutamine is an important raw material for cells to synthesize nucleic acids and proteins.

The anhydrous glucose can provide energy for cells and reduce the consumption of glycogenic amino acids.

In the cell preservation solution of the present disclosure, the combination of the compound amino acid injection, L-glutamine and anhydrous glucose has an important influence on the cell preservation effect.

A method for producing the cell preservation solution of the present disclosure comprises: according to the formula, dissolving the anhydrous glucose and L-glutamine in the compound electrolyte injection, adding the compound amino acid injection and mixing thoroughly, adding the human serum albumin and mixing evenly, adding the compound electrolyte injection to adjust the volume to 100 ml, and adjusting the pH value to 6.0-8.0 and the osmotic pressure to 275-345 mOsmol/kg.

Use of the cell preservation solution of the present disclosure in the manufacture of a mesenchymal stem cell preparation, NK cell preparation, or CART cell preparation, preferably in the manufacture of a mesenchymal stem cell preparation, NK cell preparation, or CART cell preparation that is preserved at a low temperature, wherein the low temperature is 2-8° C.

Use of the cell preservation solution of the present disclosure in the preservation of mesenchymal stem cells, NK cells, or CART cells, preferably in the preservation of mesenchymal stem cells, NK cells, or CART cells at a low temperature, wherein the low temperature is 2-8° C.

A mesenchymal stem cell preparation that is preserved at a low temperature, consisting of mesenchymal stem cells and the cell preservation solution of the present disclosure, wherein the low temperature is 2-8° C.

As a preferred embodiment of the present disclosure, the mesenchymal stem cells are prepared by the following method:
Selecting P4 generation of mesenchymal stem cells in good growth status, digesting them with trypsin to prepare a single cell suspension when the cell confluence reaches above 80%, performing centrifugation, replacing the medium with 3% HPL complete medium for culture, washing the cell pellet 2-3 times with phosphate buffered saline (PBS) after 12 h, and adjusting the cell concentration to the required concentration with the cell preservation solution of the present disclosure to obtain a mesenchymal stem cell preparation; wherein the 3% HPL complete medium is DMEM/F12 medium containing 3% HPL.

An NK cell preparation that is preserved at a low temperature, consisting of NK cells and the cell preservation solution of the present disclosure, wherein the low temperature is 2-8° C.

As a preferred embodiment of the present disclosure, the NK cells are prepared by the following method:
Selecting the NK cells in good growth status, pipetting gently and repeatedly to prepare a single cell suspension, transferring the NK cells in the culture medium into a centrifuge tube for centrifugation, washing the cell pellet 2-3 times with physiological saline, and adjusting the cell concentration to the required concentration with the cell preservation solution of the present disclosure to obtain an NK cell preparation.

A CART cell preparation that is preserved at a low temperature, consisting of CART cells and the cell preservation solution of the present disclosure, wherein the low temperature is 2-8° C.

As a preference of the present disclosure, the CART cells are prepared by the following method:
Selecting the CART cells in good growth status, pipetting gently and repeatedly to prepare a single cell suspension, transferring the CART cells in the culture medium into a centrifuge tube for centrifugation, washing the cell pellet 2-3 times with physiological saline, and adjusting the cell concentration to the required concentration with the cell preservation solution of the present disclosure to obtain a CART cell preparation.

Compared with the prior art, the present disclosure has the following advantages and effects:
1. The preservation solution of the present disclosure maintains a cell viability rate of no less than 80% for 144 h or more, greatly extending the preservation time of cell preparations and realizing the possibility of nationwide and even global application.
2. All components used in the present disclosure are clinical grade drugs and are safe and reliable for clinical application.
3. The compound amino acids in the preservation solution of the present disclosure do not contain sulfite antioxidants, which can avoid adverse reactions in patients with sensitive constitutions caused by sulfites.
4. The preservation solution of the present disclosure can realize direct intravenous infusion after preservation at a low temperature, which can avoid possible safety risks during the thawing process of the cells preserved at a low temperature, can reduce cell damage during reprocessing, and improve biological safety and cell utilization.
5. After the mesenchymal stem cells are preserved in the preservation solution of the present disclosure for 144 h, the phenotypic characteristics of the mesenchymal stem cells are still normal.
6. The preservation solution of the present disclosure contains no heparin and has a significant anti-clumping effect, with a cell clumping rate less than 12% within 288 h.
7. The cell preservation solution of the present disclosure remains stable for up to 180 days, and can be prepared into shelf products with great commercialization prospects.

The cell preservation solution of the present disclosure, as an organic whole, provides a suitable preservation environment for cells through the specific ratio of each component and the specific concentration of cells in the preservation solution, and achieves the ability to maintain corresponding cell viability rate for a long time under low temperature conditions and to maintain a low clumping rate without adding heparin. Moreover, the cell preservation solution of the present disclosure is stable and safe, and has very promising commercialization prospects.

### BRIEF DESCRIPTION OF DRAWINGS

FIG. 1 shows the detection results of cell positive phenotype by flow cytometry of mesenchymal stem cells.

FIG. 2 shows the detection results of cell negative phenotype by flow cytometry of mesenchymal stem cells.

FIG. 3 shows the detection results of positive phenotype of mesenchymal stem cells preserved for 72 h in the preservation solution of the present disclosure.

FIG. 4 shows the detection results of negative phenotype of mesenchymal stem cells preserved for 72 h in the preservation solution of the present disclosure.

FIG. 5 shows the detection results of positive phenotype of mesenchymal stem cells preserved for 144 h in the preservation solution of the present disclosure.

FIG. 6 shows the detection results of negative phenotype of mesenchymal stem cells preserved for 144 h in the preservation solution of the present disclosure.

FIG. 7 shows the stability results of amino acids in the supernatant of the cell preparation placed for 2 days.

FIG. 8 shows the stability results of amino acids in the supernatant of the cell preparation placed for 3 days.

FIG. 9 shows the stability results of amino acids in the supernatant of the cell preparation placed for 7 days.

FIG. 10 shows the stability results of human serum albumin in the supernatant of the cell preparation placed for 2 and 7 days.

FIG. 11 shows the stability results of amino acids in the lysates of the cell preparation placed for 2 days.

FIG. 12 shows the stability results of amino acids in the lysates of the cell preparation placed for 3 days.

FIG. 13 shows the stability results of amino acids in the lysates of the cell preparation placed for 7 days.

FIG. 14 shows the stability results of human serum albumin in the lysates of the cell preparation placed for 2 and 7 days.

FIG. 15 shows the stability results of amino acids after interaction for 2 days.

FIG. 16 shows the stability results of amino acids after interaction for 3 days.

FIG. 17 shows the stability results of amino acids after interaction for 7 days.

FIG. 18 shows the stability results of human serum albumin after interaction for 2, 3, and 7 days.

FIG. 19 shows the stability results of amino acids in the preservation solution (containing 14AA-SF) placed for 14 days.

FIG. 20 shows the stability results of amino acids in the preservation solution (containing 14AA-SF) placed for 28 days.

FIG. 21 shows the stability results of amino acids in the preservation solution (containing 14AA-SF) placed for 60 days.

FIG. 22 shows the stability results of amino acids in the preservation solution (containing 14AA-SF) placed for 90 days.

FIG. 23 shows the stability results of amino acids in the preservation solution (containing 14AA-SF) placed for 180 days.

FIG. 24 shows the stability results of human serum albumin in the preservation solution (containing 14AA-SF) for 14, 28, 60, 90 and 180 days.

### DETAILED DESCRIPTION

In order to facilitate those skilled in the art to better understand the present disclosure, specific embodiments of the present disclosure will be described in detail below, but it must be noted that the embodiments of the present disclosure are not limited thereto.

The sources of each reagent in the following examples are as follows:
Human serum albumin, with a specification of 10 g/50 ml, was purchased from Jetbelin Biological Products (Switzerland) Co., Ltd.
Compound electrolyte injection, with a specification of 500 ml/bag, was purchased from Baxter Medical Supplies (Shanghai) Co., Ltd.
Compound amino acid injection, with a product name of Compound amino acid injection (14AA-SF), was purchased from Halfsky Pharmacy (Hubei) Co., Ltd.
L-glutamine, with a specification of 25 kg/barrel, was purchased from Shenhua Pharmaceutical (Jiangsu). Co., Ltd.
Anhydrous glucose was purchased from Shengtai Pharmaceutical.

### Example 1

Every 100 ml of the cell preservation solution comprised 5 ml of human serum albumin preparation, 90 ml of compound electrolyte injection, 5 ml of compound amino acid injection (14AA-SF), 0.01 g of L-glutamine, and 0.1 g of anhydrous glucose.

According to the formula, the anhydrous glucose and L-glutamine were dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 6.97, and the osmotic pressure was detected to be 341 mOsmol/kg.

### Example 2

Every 100 ml of the cell preservation solution comprised 15 ml of human serum albumin preparation, 84 ml of compound electrolyte injection, 1 ml of compound amino acid injection (14AA-SF), 1 g of L-glutamine, and 1 g of anhydrous glucose.

According to the formula, the anhydrous glucose and L-glutamine were dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 7.00, and the osmotic pressure was detected to be 343 mOsmol/kg.

### Example 3

Every 100 ml of the cell preservation solution comprised 10 ml of human serum albumin preparation, 88 ml of compound electrolyte injection, 2 ml of compound amino acid injection (14AA-SF), 0.05 g of L-glutamine, and 0.5 g of anhydrous glucose.

According to the formula, the anhydrous glucose and L-glutamine were dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 6.97, and the osmotic pressure was detected to be 318 mOsmol/kg.

### Comparative Example 1

A composition of human serum albumin and compound electrolyte injection was taken as Comparative Example 1, and was prepared as follows. Every 100 ml of a cell preservation solution comprised 10 ml of human serum albumin and 90 ml of compound electrolyte injection. The pH value was adjusted to 7.04, and the osmotic pressure was detected to be 275 mOsmol/kg.

### Example 4

Preparation of Mesenchymal Stem Cell Preparation for Clinical Use
1. The P4 generation of mesenchymal stem cells in good growth status was selected. When the cell confluence reached above 80%, the cells were digested with trypsin to prepare a single cell suspension for centrifugation, and then the medium was replaced with 3% HPL complete medium for culture.
2. After 12 h, the cell pellet was washed 3 times with PBS. The cell concentration was adjusted to 5.0×10⁵ cells/ml with the preservation solution prepared in Comparative Example 1 and Examples 1-3, respectively. The cell preparation was sealed in separate packages, 20 ml/bag, and stored at 2-8° C. Cells were stained with AO/PI dyes and counted using a counter (Ruiyu Biotechnology (Shanghai) Co., Ltd., Count Star) every 24 h to detect cell viability rate. The results are shown in Table 1.

TABLE 1
Cell viability rate of mesenchymal stem cell preparations

| | | Comparative Example 1 | Example 1 | Example 2 | Example 3 |
|---|---|---|---|---|---|
| Time | | Viability rate (%) | | | |
| 0 | h | 93.67 | 94.46 | 93.65 | 94.6 |
| 24 | h | 87.9 | 93.71 | 91.92 | 94.94 |
| 48 | h | 83.44 | 93.26 | 91.65 | 94.4 |
| 72 | h | 78.26 | 91 | 88.58 | 93 |
| 96 | h | / | 89.24 | 85.95 | 88 |
| 120 | h | / | 89.7 | 83.19 | 84.94 |
| 144 | h | / | 86.39 | 80.74 | 85.62 |
| 168 | h | / | 85.14 | 78.23 | 80.63 |
| 192 | h | / | 82.99 | / | 77.71 |
| 216 | h | / | 79.31 | / | / |

Note:
“/” in the table indicates that the viability rate is less than 80%, and viability detection will no longer be performed.

The above results show that at a low temperature of 2-8° C., after mesenchymal stem cells were preserved in the preservation solution prepared in Comparative Example 1 for 48 h, the cell viability rate was above 80%. More importantly, after mesenchymal stem cells were preserved in the preservation solution prepared in Examples 1-3 for 144 h, the viability rate was still not less than 80%. This shows that the preservation solution prepared by the method of the present disclosure can effectively extend the preservation time of cells and ensure a high viability rate. It can be seen that the composition of the present disclosure plays an important role in ensuring that mesenchymal stem cells maintain high viability rate for a long time.

### Example 5

Preparation of Mesenchymal Stem Cell Preparation for Clinical Use
1. The P4 generation of mesenchymal stem cells in good growth status from excellent donor sources was selected. When the cell confluence reached above 80%, the cells were digested with trypsin to prepare a single cell suspension for centrifugation, and then the medium was replaced with 3% HPL complete medium for culture.
2. After 12 h, the cell pellet was washed 3 times with PBS. The cell concentration was adjusted to 5.0×10⁵ cells/ml with the preservation solution prepared in Comparative Example 1 and Example 1, respectively. The cell preparation was sealed in separate packages, 20 ml/bag, and stored at 2-8° C. Cells were stained with AO/PI dyes and counted using a counter (Ruiyu Biotechnology (Shanghai) Co., Ltd., Count Star) every 24 h to detect cell viability rate. The results are shown in Table 2.

TABLE 2
Cell viability rate of mesenchymal stem cell preparations

| | |
|---|---|
| Comparative Example 1 | Example 1 |

| | |
|---|---|
| Time | Viability rate (%) |

| | | | |
|---|---|---|---|
| 0 | h | 96.67 | 96.53 |
| 24 | h | 95.85 | 97.44 |
| 48 | h | 93.69 | 97.07 |
| 72 | h | 87.23 | 95.43 |
| 96 | h | 73.77 | 91.14 |
| 120 | h | / | 93.52 |
| 144 | h | / | 94.62 |
| 168 | h | / | 88.36 |
| 192 | h | / | 85.6 |
| 216 | h | / | 87.3 |
| 240 | h | / | 81.7 |
| 264 | h | / | 80.51 |
| 288 | h | / | 81.87 |

Note:
“/” in the table indicates that the viability rate is less than 80%, and viability detection will no longer be performed.

The above results show that at a low temperature of 2-8° C., after mesenchymal stem cells were preserved in the preservation solution prepared in Comparative Example 1 for 96 h, the cell viability dropped to less than 80%. More importantly, after mesenchymal stem cells were preserved in the preservation solution prepared in Example 1 for 288 h, the viability rate was still not less than 80%. This shows that the preservation solution prepared by the method of the present disclosure can effectively extend the preservation time of cells and ensure a high viability rate. In addition, there is a certain relationship between the maintenance time and the source of the donor. Excellent donor sources can prolong the maintenance time of cell viability rate to a certain extent.

### Comparative Example 2

A composition of human serum albumin and physiological saline was taken as Comparative Example 2, and was prepared as follows. Every 100 ml of a cell preservation solution comprised 25 ml of human serum albumin and 75 ml of physiological saline. The pH value was adjusted to 7.2, and the osmotic pressure was detected to be 292 mOsmol/kg.

### Example 6

Preparation of NK Cell Preparation for Clinical Use.
1. NK cells in good growth status were selected and pipetted gently and repeatedly to prepare a single cell suspension. The NK cells in the culture medium were transferred into a centrifuge tube for centrifugation.
2. After the cell pellet was washed 3 times with physiological saline, the cell concentration was adjusted to 1.0×10⁷ cells/ml with the preservation solution prepared in Comparative Example 2 and Example 1, respectively. The cell preparation was sealed in separate packages, 100 ml/bag, and stored at 2-8° C. Cells were stained with AO/PI dyes and counted using a counter (Ruiyu Biotechnology (Shanghai) Co., Ltd., Count Star) every 24 h to detect cell viability rate. The results are shown in Table 3.

TABLE 3
Cell viability rate of NK cell preparations

| | |
|---|---|
| Comparative Example 2 | Example 1 |

| | |
|---|---|
| Time | Viability rate (%) |

| | | | |
|---|---|---|---|
| 0 | h | 96.08 | 92.82 |
| 24 | h | 93.1 | 94.32 |
| 48 | h | 86.77 | 94.09 |
| 72 | h | 74.13 | 91.64 |
| 96 | h | 66.47 | 88.07 |
| 120 | h | 22.76 | 86.67 |
| 144 | h | 16.84 | 84.39 |
| 168 | h | 10.96 | 82.58 |
| 192 | h | / | 78.12 |

Note:
“/” in the table indicates that the viability rate is less than 80%, and viability detection will no longer be performed.

The above results show that at a low temperature of 2-8° C., after NK cells were preserved in the preservation solution prepared in Comparative Example 2 for 72 h, the cell viability rate dropped to less than 80%, and dropped to about 60% at 96 h, indicating that physiological saline and human serum albumin can maintain a high viability rate for a certain period of time. More importantly, however, after NK cells were preserved in the preservation solution prepared in Example 1 for 168 h, the viability rate was still not less than 80%. This shows that the composition of the present disclosure and the preservation solution prepared by the method of the present disclosure can effectively extend the preservation time of NK cells and ensure a high viability rate.

### Example 7

Preparation of CART Cell Preparation for Clinical Use
1. CART cells in good growth status were selected and pipetted gently and repeatedly to prepare a single cell suspension. The CART cells in the culture medium were transferred into a centrifuge tube for centrifugation.
2. After the cell pellet was washed 3 times with physiological saline, the cell concentration was adjusted to 2.5×10⁶ cells/ml with the preservation solution prepared in Comparative Example 2 and Example 1, respectively. The cell preparation was sealed in separate packages, 100 ml/bag, and stored at 2-8° C. Cells were stained with AO/PI dyes and counted using a counter (Ruiyu Biotechnology (Shanghai) Co., Ltd., Count Star) every 24 h to detect cell viability rate. The results are shown in Table 4.

TABLE 4
Cell viability rate of CART cell preparations

| | |
|---|---|
| Comparative Example 2 | Example 1 |

| | |
|---|---|
| Time | Viability rate (%) |

| | | | |
|---|---|---|---|
| 0 | h | 90.25 | 93.07 |
| 24 | h | 86.17 | 92.65 |
| 48 | h | 80.83 | 90.91 |
| 72 | h | 76.03 | 87.43 |
| 96 | h | / | 85.54 |
| 120 | h | / | 83.67 |
| 144 | h | / | 81.39 |
| 168 | h | / | 74.58 |

Note:
“/” in the table indicates that the viability rate is less than 80%, and viability detection will no longer be performed.

The above results show that at a low temperature of 2-8° C., after CART cells were preserved in the preservation solution prepared in Comparative Example 2 for 48 h, the cell viability rate dropped to about 80%. More importantly, after CART cells were preserved in the preservation solution prepared in Example 1 for 144 h, the viability rate was still not less than 80%. This shows that the composition of the present disclosure and the preservation solution prepared by the method of the present disclosure can effectively extend the preservation time of CART cells and ensure a high viability rate.

### Example 8

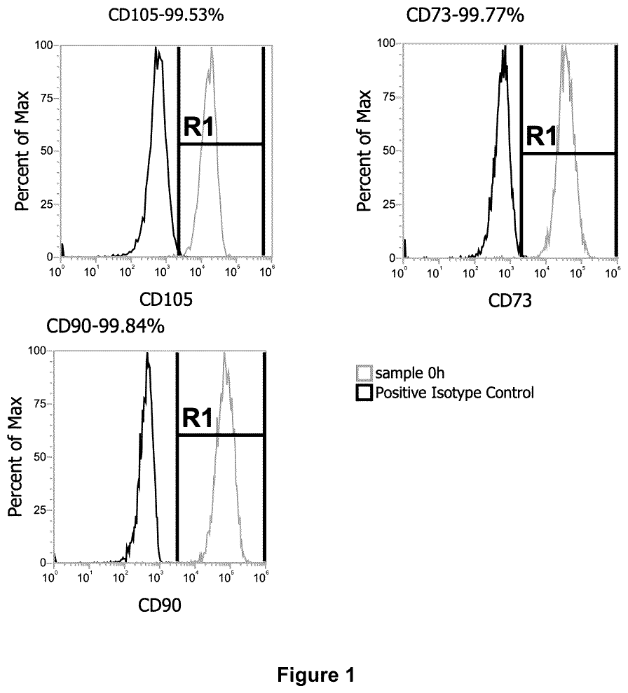

Phenotypic detection was performed on the mesenchymal stem cells that were not preserved in a cell preservation solution and the mesenchymal stem cells that were preserved in the preservation solution prepared in Example 1 for 72 and 144 h. The results are shown in FIGS. 1 to 6.

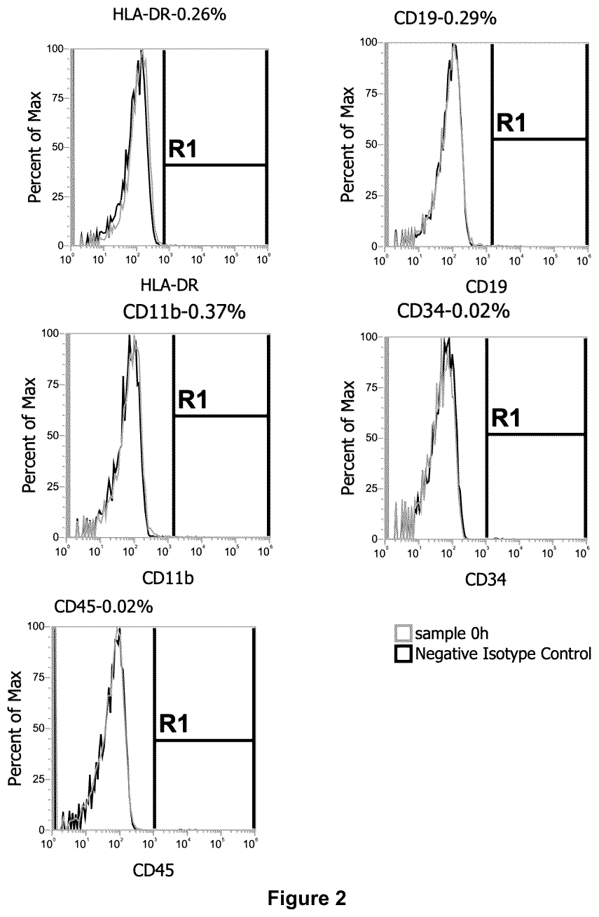

FIGS. 1 and 2 show the phenotypic detection results of mesenchymal stem cells that were not preserved in a cell preservation solution. The positive phenotypes and their expression rates were CD105-99.53%, CD73-99.77%, and CD90-99.84%, respectively. The negative phenotypes and their expression rates were HLA-DR-0.26%, CD19-0.29%, CD11b-0.37%, CD34-0.02%, and CD45-0.02%, respectively.

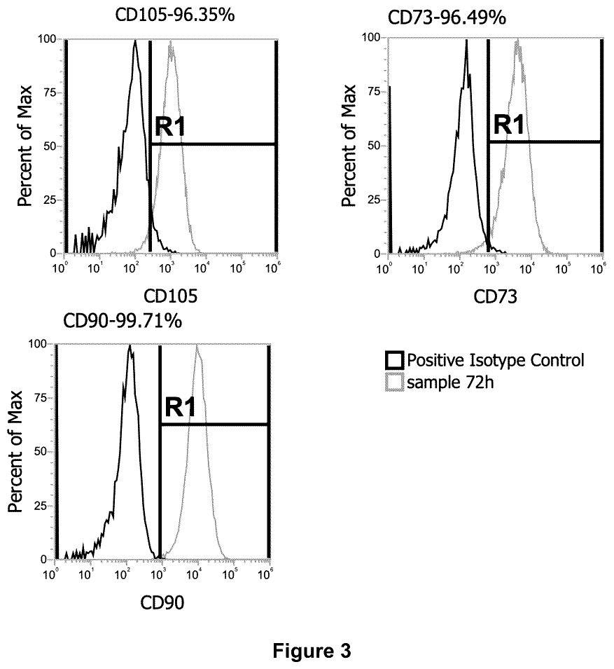

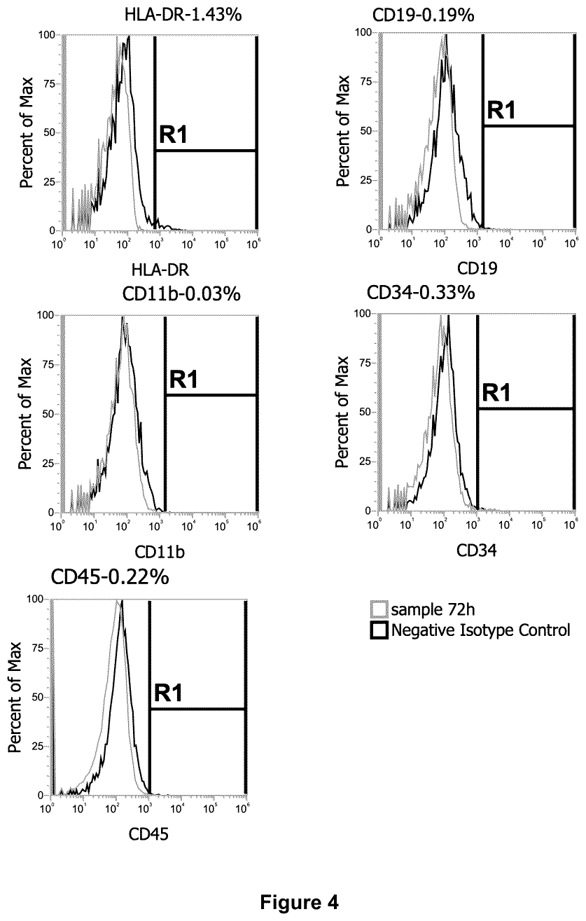

FIGS. 3 and 4 show the phenotypic detection results of mesenchymal stem cells that were preserved in the cell preservation solution for 72 h. The positive phenotypes and their expression rate were CD105-96.35%, CD73-96.49%, and CD90-99.71% respectively. The negative phenotypes and their expression rate were HLA-DR-1.43%, CD19-0.19%, CD11b-0.03%, CD34-0.33%, and CD45-0.22%, respectively.

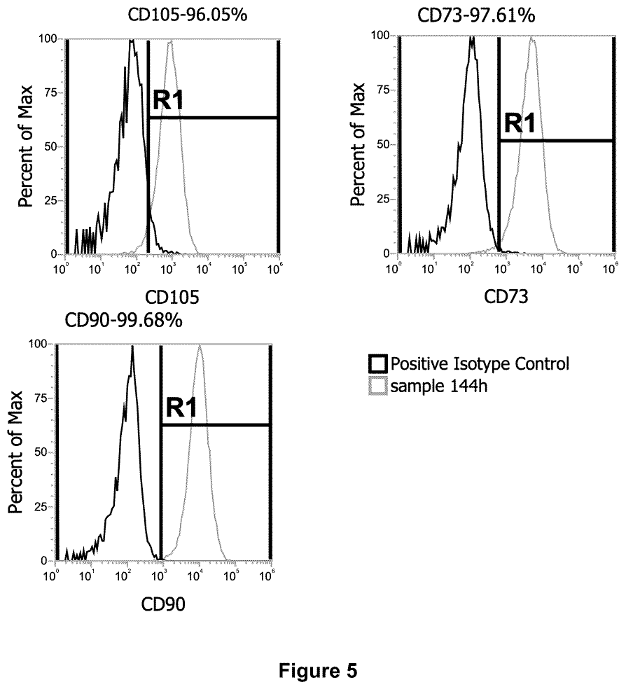

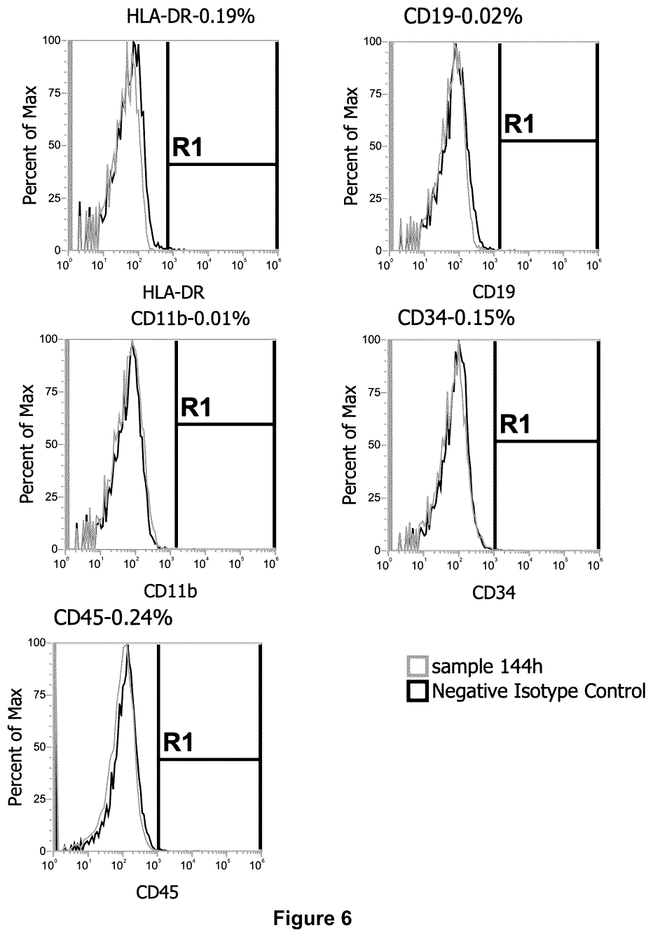

FIGS. 5 and 6 show the phenotypic detection results of mesenchymal stem cells that were preserved in the cell preservation solution for 144 h. The positive phenotypes and their expression rate were CD105-96.05%, CD73-97.61%, and CD90-99.68%, respectively. The negative phenotypes and their expression rate were HLA-DR-0.19%, CD19-0.02%, CD11b-0.01%, CD34-0.15%, and CD45-0.24%, respectively.

The expression rates of positive phenotypes CD90, CD73, and CD105 at the indicated time points were all ≥95%, and the expression rates of negative phenotypes CD19, CD34, CD45, CD11b, and HLA-DR were all ≤2%, which conformed to the phenotypic detection standards for mesenchymal stem cells, indicating that the composition of the present disclosure and the preservation solution prepared by the method of the present disclosure did not affect the phenotypic expression of mesenchymal stem cells.

### Comparative Example 3

Every 100 ml of a cell preservation solution comprised 10 ml of human serum albumin, 89 ml of compound electrolyte injection, and 1 ml of compound amino acid injection (14AA-SF).

According to the formula, the compound electrolyte injection, compound amino acid injection, and human serum albumin were added and mixed evenly, and then the compound electrolyte injection was added to adjust the volume to 100 ml. The pH value was adjusted to 7.12, and the osmotic pressure was detected to be 278 mOsmol/kg.

### Comparative Example 4

Every 100 ml of a cell preservation solution comprised 10 ml of human serum albumin, 90 ml of compound electrolyte injection, and 0.05 g of L-glutamine.

According to the formula, L-glutamine was dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 7.01, and the osmotic pressure was detected to be 276 mOsmol/kg.

### Comparative Example 5

Every 100 ml of a cell preservation solution comprised 10 ml of human serum albumin, 90 ml of compound electrolyte injection, and 0.5 g of anhydrous glucose.

According to the formula, anhydrous glucose was dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 7.03, and the osmotic pressure was detected to be 299 mOsmol/kg.

### Comparative Example 6

Every 100 ml of a cell preservation solution comprised 10 ml of human serum albumin, 89 ml of compound electrolyte injection, 1 ml of compound amino acid injection (14AA-SF), and 0.05 g of L-glutamine.

According to the formula, anhydrous glucose was dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 6.97, and the osmotic pressure was detected to be 286 mOsmol/kg.

### Comparative Example 7

Every 100 ml of a cell preservation solution comprised 10 ml of human serum albumin, 89 ml of compound electrolyte injection, 1 ml of compound amino acid injection (14AA-SF), and 0.5 g of anhydrous glucose.

According to the formula, anhydrous glucose was dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 6.98, and the osmotic pressure was detected to be 308 mOsmol/kg.

### Comparative Example 8

Every 100 ml of a cell preservation solution comprised 10 ml of human serum albumin, 90 ml of compound electrolyte injection, 0.05 g of L-glutamine, and 0.5 g of anhydrous glucose.

According to the formula, anhydrous glucose was dissolved in the compound electrolyte injection, added with the compound amino acid injection to mix thoroughly, added with the human serum albumin to mix evenly, and added with the compound electrolyte injection to adjust the volume to 100 ml. The pH value was adjusted to 7.03, and the osmotic pressure was detected to be 307 mOsmol/kg.

### Example 9

Preparation of Mesenchymal Stem Cell Preparation for Clinical Use
1. The P4 generation of mesenchymal stem cells in good growth status was selected. When the cell confluence reached above 80%, the cells were digested with trypsin to prepare a single cell suspension for centrifugation, and then the medium was replaced with 3% HPL complete medium for culture.
2. After 12 h, the cell pellet was washed 3 times with PBS. The cell concentration was adjusted to 5.0×10⁵ cells/ml with the preservation solution prepared in Comparative Example 1, Comparative Examples 3-8, and Example 1, respectively. The cell preparation was sealed in separate packages, 20 ml/bag, and stored at 2-8° C. Cells were stained with AO/PI dyes and counted using a counter (Ruiyu Biotechnology (Shanghai) Co., Ltd., Count Star) every 24 h to detect cell viability rate, clumping rate, total cell concentration, and viability. The results are shown in Table 1.
5 cells/ml with the preservation solution prepared in Comparative Example 1, Comparative Examples 3-8, and Example 1 respectively, and pack and seal in 20 ml/bag, stored at 2-8° C., stained with AO/PI dye and counted with a counter (Shanghai Ruiyu Biotechnology Co., Ltd., Count Star) every 24 hours to detect cell viability rate, clumping rate, total cell concentration, and viable cell concentration. The results are shown in Tables 5-8.

TABLE 5
Cell viability rate of mesenchymal stem cell preparations

| | | Comparative | Comparative | Comparative | Comparative | Comparative | Comparative | Comparative | |
|---|---|---|---|---|---|---|---|---|---|
| | | Example 1 | Example 3 | Example 4 | Example 5 | Example 6 | Example 7 | Example 8 | Example 1 |
| Time | | Viability rate (%) | | | | | | | |
| 0 | h | 96.67 | 96.62 | 96.3 | 97.56 | 96.05 | 96.85 | 97.22 | 96.53 |
| 24 | h | 95.85 | 92.65 | 91.83 | 90.59 | 95.7 | 96.28 | 93.13 | 97.44 |
| 48 | h | 83.69 | 90.23 | 87.46 | 84.98 | 96.85 | 96.26 | 90.14 | 97.07 |
| 72 | h | 77.23 | 88.67 | 80.22 | 80.42 | 94.84 | 95.88 | 85.24 | 95.43 |
| 96 | h | / | 84.93 | 74.35 | 72.75 | 82.26 | 85.38 | 78.97 | 93.14 |
| 120 | h | / | 78.7 | / | / | 75.54 | 74.5 | / | 90.52 |
| 144 | h | / | / | / | / | / | / | / | 87.62 |
| 168 | h | / | / | / | / | / | / | / | 83.36 |
| 192 | h | / | / | / | / | / | / | / | 77.3 |

Note:
“/” in the table indicates that the viability rate is less than 80%, and viability detection will no longer be performed.

TABLE 6
Cell clumping rate of mesenchymal stem cell preparations

| | | Comparative | Comparative | Comparative | Comparative | Comparative | Comparative | Comparative | |
|---|---|---|---|---|---|---|---|---|---|
| | | Example 1 | Example 3 | Example 4 | Example 5 | Example 6 | Example 7 | Example 8 | Example 1 |
| Time | | Clumping rate (%) | | | | | | | |
| 0 | h | 8.94 | 8.98 | 7.17 | 7.74 | 12.38 | 7.01 | 8.33 | 9.92 |
| 24 | h | 9.36 | 8.99 | 9.96 | 13.7 | 8.77 | 10.9 | 7.68 | 7.15 |
| 48 | h | 15 | 14.2 | 13.07 | 9.92 | 10.79 | 12.68 | 13.08 | 7.82 |
| 72 | h | 11.36 | 18.21 | 8.5 | 7.82 | 6.28 | 10.9 | 15.1 | 9.37 |
| 96 | h | / | 12.91 | 8.44 | 12.65 | 7.92 | 8.98 | 9.69 | 11.19 |
| 120 | h | / | 18.27 | / | / | 16.21 | 14.54 | / | 11.69 |
| 144 | h | / | / | / | / | / | / | / | 10.58 |
| 168 | h | / | / | / | / | / | / | / | 5.18 |
| 192 | h | / | / | / | / | / | / | / | 8.1 |

Note:
“/” in the table indicates that the viability rate is less than 80%, and clumping rate detection will no longer be performed.

TABLE 7
Total cell concentration of mesenchymal stem cell preparations

| | | Comparative | Comparative | Comparative | Comparative | Comparative | Comparative | Comparative | |
|---|---|---|---|---|---|---|---|---|---|
| | | Example 1 | Example 3 | Example 4 | Example 5 | Example 6 | Example 7 | Example 8 | Example 1 |
| Time | | Total cell concentration (cells/ml) | | | | | | | |
| 0 | h | 5.69E+05 | 5.82E+05 | 6.50E+05 | 5.97E+05 | 6.64E+05 | 6.70E+05 | 6.02E+05 | 5.67E+05 |
| 24 | h | 5.62E+05 | 6.10E+05 | 6.43E+05 | 6.28E+05 | 6.10E+05 | 6.07E+05 | 6.39E+05 | 5.13E+05 |
| 48 | h | 6.00E+05 | 6.56E+05 | 5.72E+05 | 5.51E+05 | 6.02E+05 | 5.45E+05 | 6.34E+05 | 3.98E+05 |
| 72 | h | 6.16E+05 | 6.70E+05 | 5.66E+05 | 5.07E+05 | 6.21E+05 | 5.66E+05 | 6.36E+05 | 3.98E+05 |
| 96 | h | / | 4.60E+05 | 5.03E+05 | 5.40E+05 | 5.94E+05 | 5.22E+05 | 5.68E+05 | 4.68E+05 |
| 120 | h | / | 5.64E+05 | / | / | 6.71E+05 | 5.86E+05 | / | 5.51E+05 |
| 144 | h | / | / | / | / | / | / | / | 4.06E+05 |
| 168 | h | / | / | / | / | / | / | 1 | 5.00E+05 |
| 192 | h | / | / | / | / | / | / | / | 4.45E+05 |

Note:
“/” in the table indicates that the viability rate is less than 80%, and total cell concentration detection will no longer be performed.

TABLE 8
Viable cell concentration of mesenchymal stem cell preparations

| | | Comparative | Comparative | Comparative | Comparative | Comparative | Comparative | Comparative | |
|---|---|---|---|---|---|---|---|---|---|
| | | Example 1 | Example 3 | Example 4 | Example 5 | Example 6 | Example 7 | Example 8 | Example 1 |
| Time | | Viable cell concentration (cells/ml) | | | | | | | |
| 0 | h | 5.50E+05 | 5.62E+05 | 6.26E+05 | 5.83E+05 | 6.38E+05 | 6.49E+05 | 5.86E+05 | 5.48E+05 |
| 24 | h | 5.39E+05 | 5.89E+05 | 6.23E+05 | 6.00E+05 | 5.84E+05 | 5.85E+05 | 6.15E+05 | 5.00E+05 |
| 48 | h | 5.62E+05 | 6.38E+05 | 5.58E+05 | 5.24E+05 | 5.83E+05 | 5.24E+05 | 5.97E+05 | 3.87E+05 |
| 72 | h | 5.38E+05 | 6.34E+05 | 5.11E+05 | 4.80E+05 | 5.88E+05 | 5.43E+05 | 5.93E+05 | 3.80E+05 |
| 96 | h | / | 4.36E+05 | 4.24E+05 | 4.88E+05 | 5.54E+05 | 4.72E+05 | 5.05E+05 | 4.27E+05 |
| 120 | h | / | 5.34E+05 | / | / | 6.31E+05 | 5.40E+05 | / | 5.15E+05 |
| 144 | h | / | / | / | / | / | / | / | 3.85E+05 |
| 168 | h | / | / | / | / | / | / | / | 4.42E+05 |
| 192 | h | / | / | / | / | / | / | / | 3.81E+05 |

Note:
“/” in the table indicates that the viability rate is less than 80%, and viable cell concentration detection will no longer be performed.

The above results show that at a low temperature of 2-8° C., mesenchymal stem cells in the cell preservation solution prepared in Comparative Example 1 can maintain a cell viability rate of 80% for 48 h, mesenchymal stem cells in the cell preservation solution prepared in Comparative Examples 4, 5, and 8 can maintain a cell viability rate of 80% for 72 h, and mesenchymal stem cells in the cell preservation solution prepared in Comparative Examples 3, 6, and 7 can maintain a cell viability rate of 80% for 96 h. This shows that with the addition of components in the composition, the viability rate of cells increased, and the addition of compound amino acids was crucial to maintaining the viability rate. In addition, the clumping rate of Comparative Examples 1 and 3 was higher than that of other groups, indicating that anhydrous glucose and L-glutamine had a certain effect on improving the clumping rate. More importantly, after preservation in the cell preservation solution prepared in Example 1 for 288 h, the viability rate can still be maintained at 80% and the clumping rate less than 12%, indicating that the composition of the present disclosure and the preservation solution prepared by the method of the present disclosure had a most significant effect in maintaining high cell viability rate and low clumping rate. Similarly, each of the above sets of data shows that the above preservation solution prepared by the method of the present disclosure can effectively maintain high cell viability rate and low clumping rate. Moreover, the total cell concentration and viable cell concentration of each group with a viability rate above 80% can meet the quality control standards (the effective range of total cell concentration was no less than 4.0E+05 cells/ml, and the effective range of viable cell concentration was no less than 3.2E+05 cells/ml), indicating that each set of values that meets the requirements was an effective amount.

### Example 10

To verify the stability of the cell preservation solution containing cells, LC-MS was used to verify the changes in human serum albumin and amino acid content in the cell supernatant, and whether cell disruption had an impact on the stability of the cell preservation solution.

1. Processing of Cell Supernatant Sample:

The cell preparation was prepared according to the formula of Example 1 and placed for 2 days (2 d). A fresh cell preparation (0 d) was prepared as a control. 700 μl of the cell preparation suspension was subjected to centrifugation at 1800 rpm for 10 min. 600 μl of the supernatant was taken, and then 100 μl of each was transferred in six 1.5 ml EP tubes. The 3 d and 7 d samples were processed in the same way.

Amino acid pretreatment: 100 μl of each sample was added with 500 μl of acetonitrile and 10 μl of internal standard, vortexed for 5 min, and centrifuged at 1800 rpm for 10 min. 450 μl of the supernatant was taken, evaporated to dryness, and redissolved for sample injection.

Human serum albumin pretreatment: albumin was quantified using the BCA method. 100 μg of protein was subjected to enzyme digestion followed by solid phase extraction, evaporated to dryness, and redissolved for sample injection.

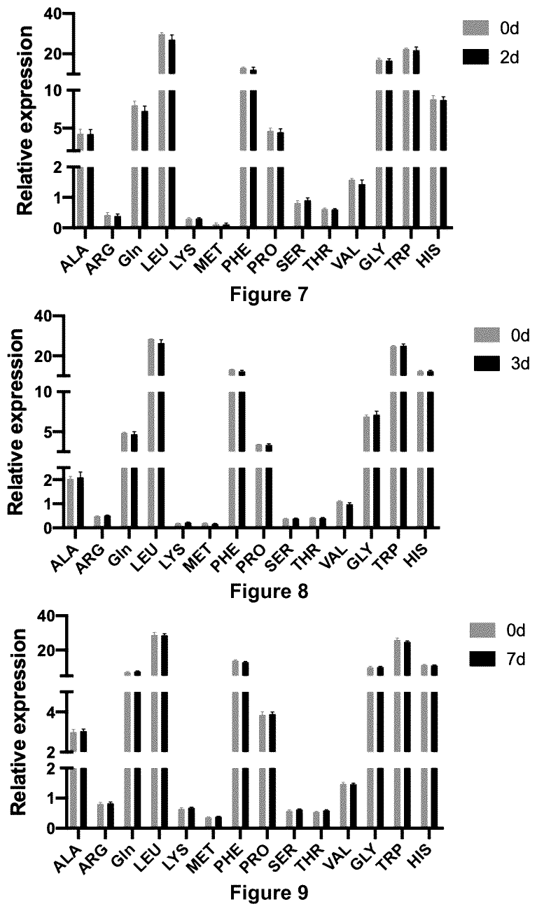

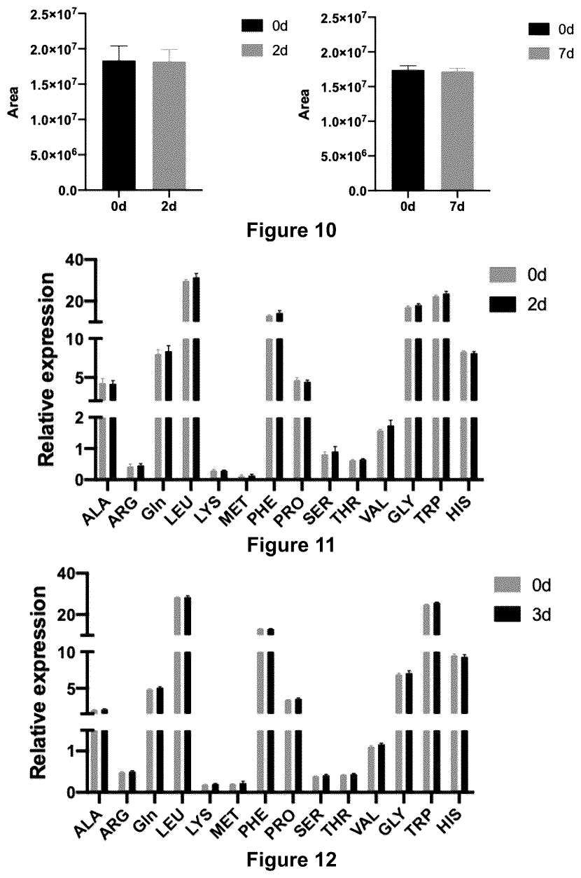

The results are shown in FIGS. 7-10. The above results show that after the preservation solution containing cells was placed at a low temperature of 2-8° C. for 2, 3, and 7 days, amino acids and human serum albumin were stable without degradation.

2. Processing of Cell Lysate Sample:

The cell preparation was prepared according to the formula of Example 1 and placed for 2 d. A fresh cell preparation (Od) was prepared as a control. 700 μl of the cell preparation suspension was subjected to ultrasonication on ice and centrifugation at 1800 rpm for 10 min. 600 μl of the supernatant was taken, and then 100 μl of each was transferred in six 1.5 ml EP tubes. The 3 d and 7 d samples were processed in the same way.

Amino acid pretreatment: 100 μl of each sample was added with 500 μl of acetonitrile and 10 μl of internal standard, vortexed for 5 min, and centrifuged at 1800 rpm for 10 min. 450 μl of the supernatant was taken, evaporated to dryness, and redissolved for sample injection.

Human serum albumin pretreatment: albumin was quantified using the BCA method. 100 μg of protein was subjected to enzyme digestion followed by solid phase extraction, evaporated to dryness, and redissolved for sample injection.

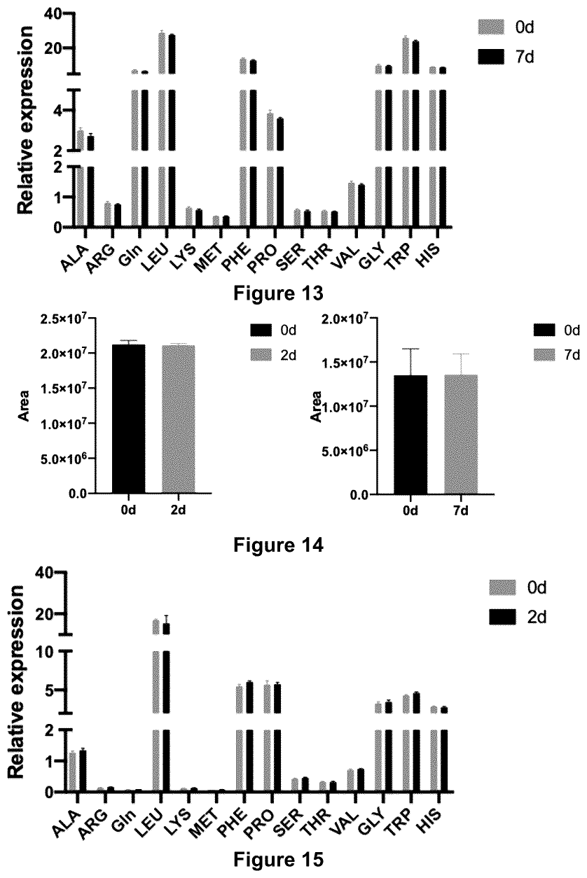

The results are shown in FIGS. 11-14. The preservation solution containing cells was placed at a low temperature of 2-8° C. for 2, 3, and 7 days, and then the cells were lysed. The above results show that even if the cells were disrupted, the stability of amino acids and albumin was not affected.

In summary, it is fully demonstrated that the storage environment of mesenchymal stem cells in the cell preservation solution was extremely stable, and the amino acids and human serum albumin can be stable for up to 7 days without degradation.

### Example 11

In order to examine the impact of cell culture residues on the cell preservation solution, the interaction between the cell preservation solution of Example 1 without cells and PBS, HPL, and DMEM/F12 was examined, and the changes in human serum albumin and amino acid content were mainly detected.

Reagent Processing:

PBS, HPL, or DMEM/F12 was mixed with the cell preservation solution at a volume ratio of 1:10, and samples were taken to detect amino acids and albumin on 0, 2, 3, and 7 days.

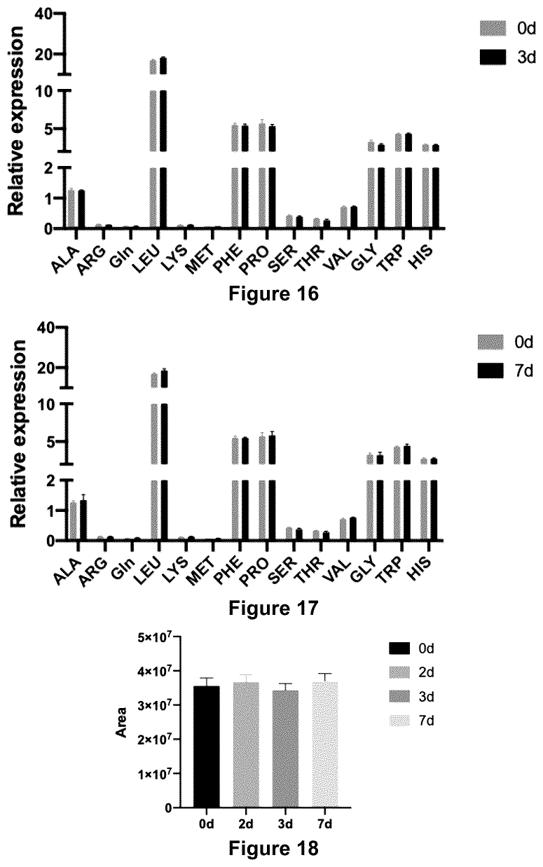

The results are shown in FIGS. 15-18. The above results show that human serum albumin and amino acids had good stability in the interaction investigation of 2, 3, and 7 days, indicating that PBS, HPL, and DMEM/F12 had no influence on the cell preservation solution.

### Example 12

To verify the long-term stability of the cell preservation solution without cells, the changes in human serum albumin and amino acid content in the preservation solution were mainly detected.

According to Example 1, four cell preservation solutions were prepared and placed for 180, 90, 60, 28, and 14 days, and 100 μl of each was transferred in five 1.5 ml EP tubes.

Amino acid pretreatment: 100 μl of each sample was added with 500 μl of acetonitrile and 10 μl of internal standard, vortexed for 5 min, and centrifuged at 1800 rpm for 10 min. 450 μl of the supernatant was taken, evaporated to dryness, and redissolved for sample injection.

Human serum albumin pretreatment: albumin was quantified using the BCA method. 100 μg of protein was subjected to enzyme digestion followed by solid phase extraction, evaporated to dryness, and redissolved for sample injection.

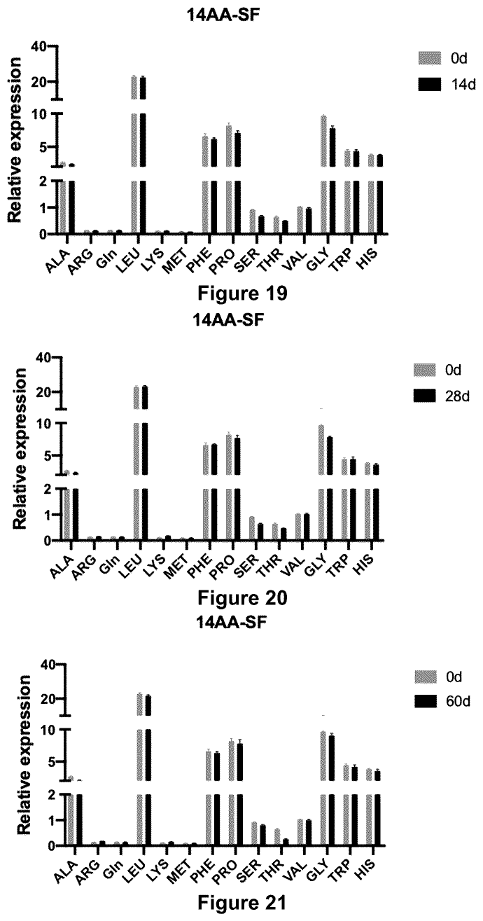

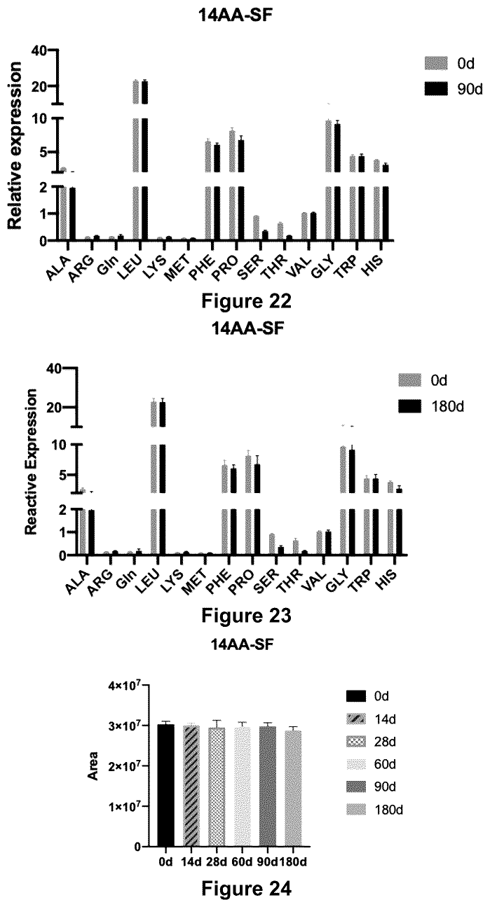

The results are shown in FIGS. 19-24. After the cell preservation solution containing 14AA-SF was placed at a low temperature of 2-8° C. for 14, 28, 60, 90, and 180 days, human serum albumin and amino acids were stable without degradation, indicating that the validity period of the cell preservation solution of the present disclosure can be as long as 180 days.

### Example 13

The stability of the cell preservation solution of Example 1 without cells was further investigated, and the influencing factor test and acceleration test about stability were conducted respectively.

The influencing factor test about stability includes high temperature, high humidity, and strong light irradiation tests. Since the cell preservation solution of the present invention is a liquid preparation, there is no need to examine high humidity tests.

The results of the strong light irradiation test and high temperature test are as follows:

TABLE 9
Influencing factor test data (50 mL)

| | Limit | Before | Light irradiation (4500LX) | | | High temperature (60° C.) | | |
|---|---|---|---|---|---|---|---|---|
| Items | requirements | placing | 5 days | 10 days | 30 days | 5 days | 10 days | 30 days |
| Traits | Colorless | Colorless | Colorless | Colorless | Colorless | Colorless | Colorless | Colorless |
| | or almost | clear liquid | clear liquid | clear liquid | clear liquid | clear liquid | clear liquid | clear liquid |
| | colorless | | | | | | | |
| | clear liquid | | | | | | | |
| pH value | 6.0-8.0 | 7.00 | 7.02 | 7.04 | 7.03 | 7.03 | 7.01 | 7.02 |
| Transmittance | ≥97.0% | 99.9 | 99.4 | 98.8 | 98.4 | 99.4 | 98.8 | 98.4 |
| Insoluble | ≥10 um 6000 | 2 | 1 | 4 | 3 | 1 | 4 | 4 |
| particles | particles | | | | | | | |
| | ≥25 um 600 | 1 | 0 | 0 | 0 | 0 | 1 | 1 |
| | particles | | | | | | | |
| Acetic acid | 90-110 | 97.1 | 98.0 | 98.5 | 99.4 | 98.9 | 99.2 | 99.8 |
| | mmol/L | | | | | | | |
| Visible | Should | Meeting | Meeting | Meeting | Meeting | Meeting | Meeting | Meeting |
| foreign | meet the | the | the | the | the | the | the | the |
| matter | requirements | requirements | requirements | requirements | requirements | requirements | requirements | requirements |
| Isoleucine | 90.0-110.0% | 99.1 | 99.0 | 98.9 | 98.4 | 98.7 | 98.5 | 98.4 |
| Leucine | of the | 99.4 | 99.3 | 99.3 | 99.4 | 9.1 | 98.9 | 98.6 |
| Lysine acetate | indicated | 100.7 | 100.1 | 100.0 | 99.8 | 99.9 | 99.6 | 99.2 |
| Methionine | amount | 99.0 | 99.0 | 98.7 | 98.7 | 98.6 | 98.3 | 98.3 |
| Phenylalanine | | 101.2 | 100.8 | 100.4 | 99.6 | 100.5 | 100.1 | 99.9 |
| Threonine | | 98.2 | 98.2 | 98.1 | 97.8 | 97.9 | 97.4 | 97.3 |
| Tryptophan | | 99.3 | 99.2 | 98.7 | 98.4 | 99.3 | 99.0 | 98.2 |
| Valine | | 99.3 | 99.1 | 98.7 | 98.7 | 98.8 | 98.4 | 98.4 |
| Alanine | | 98.6 | 98.4 | 98.3 | 97.7 | 98.1 | 98.0 | 97.0 |
| Arginine | | 99.0 | 98.8 | 98.5 | 98.7 | 98.7 | 98.2 | 98.0 |
| Histidine | | 100.6 | 100.3 | 99.8 | 99.4 | 99.9 | 99.7 | 98.9 |
| Proline | | 100.5 | 100.2 | 100.2 | 100.3 | 100.2 | 100.1 | 99.4 |
| Serine | | 97.3 | 97.2 | 97.1 | 97.1 | 97.1 | 96.7 | 96.4 |
| Glycine | | 100.4 | 100.0 | 99.9 | 99.6 | 99.9 | 99.7 | 99.8 |
| Human serum | | 99.3 | 98.9 | 98.4 | 98.1 | 98.5 | 98.2 | 97.7 |
| albumin | | | | | | | | |
| Glucose | | 98.7 | 98.5 | 98.3 | 98.0 | 98.3 | 98.0 | 97.5 |
| L-Glutamine | | 100.1 | 99.7 | 99.6 | 99.4 | 99.5 | 98.9 | 98.0 |
| Sodium | | 99.7 | 98.9 | 98.1 | 97.5 | 99.5 | 98.9 | 98.2 |
| chloride | | | | | | | | |
| Sodium | | 99.7 | 99.5 | 99.2 | 98.8 | 99.4 | 99.0 | 98.4 |
| gluconate | | | | | | | | |
| Sodium | | 99.6 | 99.1 | 98.5 | 97.9 | 99.0 | 98.2 | 97.4 |
| acetate | | | | | | | | |
| Potassium | | 100.2 | 100.0 | 99.6 | 99.2 | 99.8 | 99.2 | 98.5 |
| chloride | | | | | | | | |
| Magnesium | | 98.9 | 98.5 | 98.1 | 97.5 | 98.0 | 97.2 | 96.2 |
| chloride | | | | | | | | |

The “Chinese Pharmacopoeia” states that the test sample for acceleration test should be placed for 6 months at a temperature of 40° C.±2° C. and a relative humidity of 75%±5%. The cell preservation solution prepared by the present disclosure was sampled and tested at 0 days, 1 month, 2 months, 3 months, and 6 months under these conditions.

The acceleration test results are as follows:

TABLE 10
Acceleration test data

| | Limit | | | | | |
|---|---|---|---|---|---|---|
| Items | requirements | 0 day | 1 month | 2 months | 3 months | 6 months |
| Traits | Colorless or | Colorless | Colorless | Colorless | Colorless | Colorless |
| | almost colorless | clear | clear | clear | clear | clear |
| | clear liquid | liquid | liquid | liquid | liquid | liquid |
| pH value | 6.0-8.0 | 7.00 | 7.02 | 7.04 | 7.03 | 7.03 |
| Transmittance | ≥97.0% | 99.9 | 99.5 | 99.2 | 99.0 | 98.8 |
| Insoluble | ≥10 um 6000 | 2 | 3 | 3 | 4 | 4 |
| particles | particles | | | | | |
| | ≥25 um 600 | 1 | 1 | 0 | 1 | 0 |
| | particles | | | | | |
| Acetic acid | 90-110 | 98.0 | 98.5 | 99.6 | 99.5 | 99.8 |
| | mmol/L | | | | | |
| Bacterial | <0.5 | Meeting the | / | / | / | Meeting the |
| endotoxin | EU/ml | requirements | | | | requirements |
| Visible foreign | Should meet the | Meeting the | Meeting the | Meeting the | Meeting the | Meeting the |
| matter | requirements | requirements | requirements | requirements | requirements | requirements |
| Sterile | Should meet the | Meeting the | / | / | / | Meeting the |
| | requirements | requirements | | | | requirements |
| Isoleucine | 90.0-110.0% of | 99.1 | 100.4 | 100.7 | 100.8 | 99.8 |
| Leucine | the indicated | 99.4 | 100.5 | 101.0 | 101.4 | 101.0 |
| Lysine acetate | amount | 100.7 | 99.5 | 99.5 | 99.8 | 99.8 |
| Methionine | | 99.0 | 100.8 | 100.0 | 100.0 | 99.8 |
| Phenylalanine | | 101.2 | 99.8 | 99.5 | 99.5 | 99.5 |
| Threonine | | 98.2 | 99.3 | 100.6 | 100.5 | 99.9 |
| Tryptophan | | 99.3 | 99.4 | 99.4 | 99.9 | 99.9 |
| Valine | | 99.3 | 99.8 | 100.3 | 100.1 | 99.6 |
| Alanine | | 98.6 | 99.8 | 100.7 | 100.3 | 100.1 |
| Arginine | | 99.0 | 101.6 | 100.9 | 100.6 | 100.5 |
| Histidine | | 100.6 | 99.3 | 100.1 | 99.2 | 99.1 |
| Proline | | 100.5 | 101.4 | 100.7 | 100.1 | 100.2 |
| Serine | | 97.3 | 99.7 | 101.2 | 101.1 | 100.0 |
| Glycine | | 100.4 | 100.3 | 101.3 | 100.9 | 100.3 |
| Human serum | | 99.3 | 99.5 | 99.2 | 98.8 | 98.5 |
| albumin | | | | | | |
| Glucose | | 98.7 | 98.5 | 98.2 | 98.0 | 97.6 |
| L-Glutamine | | 100.1 | 99.7 | 99.2 | 99.0 | 98.5 |
| Sodium | | 99.7 | 98.9 | 98.0 | 97.1 | 96.5 |
| chloride | | | | | | |
| Sodium | | 99.7 | 99.5 | 99.0 | 98.4 | 97.4 |
| gluconate | | | | | | |
| Sodium acetate | | 99.6 | 99.1 | 98.2 | 97.3 | 96.5 |
| Potassium | | 100.2 | 100.0 | 99.5 | 99.1 | 98.6 |
| chloride | | | | | | |
| Magnesium | | 98.9 | 98.4 | 97.7 | 97.1 | 96.3 |
| chloride | | | | | | |

The above results show that the components of the cell preservation solution were stable after being placed under strong light irradiation and high temperature conditions for 30 days, and the components were still stable without degradation after 6 months of acceleration test, indicating that the cell preservation solution of the present disclosure had good stability.

1. A composition for producing a cell preservation solution, consisting of a compound amino acid injection, L-glutamine and anhydrous glucose at a ratio of (1-5) ml:(0.01-1) g:(0.1-1) g.

2. The composition according to claim 1, wherein the compound amino acid injection is compound amino acid injection 14AA, preferably sulfite antioxidant-free compound amino acid injection 14AA, 14AA-SF.

3. A cell preservation solution comprising the composition according to claim 1, wherein preferably, every 100 ml of the cell preservation solution comprises 5-15 ml of human serum albumin preparation, 80-90 ml of compound electrolyte injection, 1-5 ml of sulfite antioxidant-free compound amino acid injection, 0.01-1 g of L-glutamine, and 0.1-1 g of anhydrous glucose.

4. The cell preservation solution according to claim 3, wherein the sulfite antioxidant-free compound amino acid injection is sulfite antioxidant-free compound amino acid injection 14AA, 14AA-SF.

5. Application of the cell preservation solution according to claim 3 in the manufacture of a mesenchymal stem cell preparation, NK cell preparation, or CART cell preparation, preferably in the manufacture of a mesenchymal stem cell preparation, NK cell preparation, or CART cell preparation that is preserved at a low temperature, wherein the low temperature is 2-8° C.

6. Application of the cell preservation solution according to claim 3 in the preservation of mesenchymal stem cells, NK cells, or CART cells, preferably in the preservation of mesenchymal stem cells, NK cells, or CART cells at a low temperature, wherein the low temperature is 2-8° C.

7. The cell preservation solution according to claim 3, further comprising mesenchymal stem cells, which is preserved at a low temperature, wherein the low temperature is 2-8° C.

8. The cell preservation solution according to claim 3, further comprising NK cells, which is preserved at a low temperature, wherein the low temperature is 2-8° C.

9. The cell preservation solution according to claim 3, further comprising CART cells, which is preserved at a low temperature, wherein the low temperature is 2-8° C.
